(19) United States
(12) Patent Application Publication
(10) Pub. No.: US 2022/0362382 A1
(43) Pub. Date: Nov. 17, 2022

(54) TOPICAL COMPOSITION COMPRISING A PROSTAGLANDIN ANALOGUE

(71) Applicant: DERMALIQ THERAPEUTICS, INC, Wilmington, DE (US)

(72) Inventors: Frank LÖSCHER, Schriesheim (DE); Bernhard HAUPTMEIER, Schriesheim (DE)

(21) Appl. No.: 17/634,184

(22) PCT Filed: Aug. 6, 2020
(86) PCT No.: PCT/EP2020/072211

§ 371 Date: Feb. 9, 2022

(30) Foreign Application Priority Data
Aug. 9, 2019 (EP) 19191035.5

(51) Int. Cl. A61K 47/06; A61K 9/00; A61K 31/5575; A61K 47/10; A61P 17/14; A61K 9/08

(52) U.S. Cl. CPC A61K 47/06; A61K 9/0014; A61K 31/5575; A61K 47/10; A61P 17/14; A61K 9/08

(57) ABSTRACT

The present invention relates to a topical composition useful for the treatment of hair disorders such as androgenetic alopecia, hirsutism, alopecia of the eyebrows and eyelashes, and hypotrichosis.

The present invention relates to topical compositions which are useful for the treatment of diseases and conditions affecting a pilosebaceous unit, in particular for the treatment of hair disorders.

### BACKGROUND ART

A pilosebaceous unit is composed of the hair follicle, the hair shaft and sebaceous glands. This structure is present on the surface of the mammalian skin and is considered to be an important pathway for the percutaneous absorption of topically applied drugs. Within the skin the pilosebaceous unit is the main factory for hormone production, in particular can synthesize androgens. When stimulated by hormones such as androgens, sebaceous glands secrete a lipid-rich sebum that protects the hair and provides the skin with a hydrophobic barrier that can serve as protection.

The hair follicle is an invagination of the epidermis extending deep into the dermis. Targeted drug delivery to hair follicle is relevant with regard to diseases like androgenetic alopecia and alopecia areata. Access to the hair follicle is difficult due to structural aspect of hair follicle and its chemical environment. The keratinous layers of the inner and outer root sheaths and the glassy membrane surrounding the entire follicle may restrict passage of molecules deep within the follicle. Further, sebum discharge into the follicle is constant and effective drug delivery and pharmacological effects depend upon the interactions between the drug and sebum. (Indian Journal of Pharmacology 2000; 32: 269-281).

An androgen-dependent condition, disease, disorder, or syndrome, is a medical condition that is, in part or full, dependent on, or is sensitive to, the presence of androgenic activity in the body. Known androgen-dependent conditions include among others androgenic alopecia and hirsutism. Alopecia, also known as hair loss or baldness, refers to the loss of hair from parts of the head or body. Hirsutism refers to the male pattern hair distribution in women. Both conditions cause significant psychologic distress.

Androgenetic alopecia (also known as androgenic alopecia) affects both men and women. In men it produces male pattern hair loss with bitemporal recession and vertex baldness. In women it produces female pattern hair loss with diffuse alopecia over the mid-frontal scalp.

Androgen dependent conditions may be treated with drugs with antiandrogen actions, including androgen receptor antagonists such as cyproterone acetate, spironolactone, and bicalutamide, 5α-reductase inhibitors such as finasteride and dutasteride, CYP17A1 inhibitors, gonadotropin-releasing hormone (GnRH) analogues and/or other antigonadotropins. Topical minoxidil and oral finasteride are approved by the Food and Drug Administration (USA) for the treatment of male androgenetic alopecia. Both medications prevent further hair loss, but only partially reverse baldness, and require continuous use to maintain the effect.

Hirsutism is excessive body hair on parts of the body where hair is normally absent or minimal. Classically, hirsutism has been considered a marker of increased androgen levels in females from increased production of androgens (i.e testosterone) either by the adrenals or due to an ovarian disease. Spironolactone (SPA) is an androgen blocker. The starting dose is 50 mg twice daily and may be increased to a total daily dose of 200 mg. The 5-RA inhibitors finasteride, a 5-alpha reductase inhibitor, has been found to be effective in the treatment of hirsutism. (Indian J Dermatol. 2010 January-March; 55(1): 3-7).

Physiologically, eyelashes serve a protective function and, as with all hairs on the body, are generated by the continuous cycling of hair follicles. Similar to other hair follicles on the body, eyelash follicles are connected to sebaceous glands. Individuals can experience the loss of previously normal eyelashes, which may or may not be accompanied by the destruction of their respective hair follicles. This condition, madrosis (also referred to as milphosis), can have numerous causes, including alopecia areata, infection, endocrine disease, like hypothyroidism, medications, radiation, or trauma.

Hypotrichosis is a rare condition in which there is little or no hair growth on the head, including the brows above the eyes and the edge of the eyelids, or other areas of the body where hair normally grows.

EP2802331B1 describes bimatoprost 0.03% w/v for use in a method of growing eyelashes in post chemotherapeutic patients.

When prescribed for the treatment of eyelash hypotrichosis, bimatoprost ophthalmic solution 0.03% is to be applied daily to the skin of the upper eyelid margin at the base of the eyelashes using a sterile single-use-per-eye applicator.

Latanoprost is an isopropyl ester prodrug of the acid metabolite, which is a prostaglandin F2α analog. U.S. Pat. No. 6,262,105B1 describes methods of enhancing hair growth using prostaglandins, specifically exemplifying latanoprost. This patent also describes topical preparations having varying amounts (0.1-10%) of active ingredient.

Blume-Peytavi (J. Am. Acad. Dermatol. 2012 May; 66(5):794-800) describes a double-blind placebo-controlled pilot study to assess the efficacy of a 24-week topical treatment by latanoprost 0.1% on hair growth and pigmentation in healthy volunteers with androgenetic alopecia. The aqueous latanoprost formulation (0.1% latanoprost, 50% ethanol, 20% propylene glycol, water) used in the study comprises high amount of ethanol which can provoke unpleasant reactions and inflammation, especially when applied to sensitive areas like the eyelids.

It is therefore an object of the present invention to provide an effective topical treatment of a disease or condition affecting a pilosebaceous unit such as androgenetic alopecia, hirsutism, hypotrichosis, alopecia of the eyebrows and of the eyelashes. A further object is to provide a treatment which effectively delivers prostaglandins or antiandrogens to the hard to reach pilosebaceous unit or components thereof, namely to or into the hair follicle, the hair shaft and sebaceous glands. In a further aspect, it is an objective of the invention to provide pharmaceutical compositions that contain cosolvents, such as alcohols, at a significant lower concentration, as compared to aqueous compositions, which utilize alcohols in higher concentrations as penetration enhancers and which thus may lead to skin irritation or contact dermatitis, when used on a regular basis. These and further objects will become clear on the basis of the description of the invention and the patent claims.

### SUMMARY OF THE INVENTION

The objective of the present invention is to provide a pharmaceutical composition which may be used to treat or prevent a disease or condition affecting a pilosebaceous unit such as androgenetic alopecia, hirsutism, hypotrichosis, alopecia of the eyebrows and of the eyelashes. The objective of the present invention is attained by the claims. The composition of the present invention penetrates effectively into the skin and the hairs, preferably into the pilosebaceous unit of the skin and the hairs or components thereof, such as the hair follicle, the hair shaft and sebaceous glands of the skin and the hairs. Accordingly, treatment of diseases associated with the pilosebaceous unit, in particular diseases of the hair, can be conveniently achieved by topical application and without the side effects associated with the use of water based formulations, characterized by high amounts of cosolvents and other components which can be irritating, in particular for delicate areas like the eyelids.

### DESCRIPTION OF THE DRAWINGS

FIG. 1: Uptake of latanoprost from latanoprost formulations after 2 hours of incubation. The concentration of latanoprost is expressed in μg per cm² of skin. Exp1 and Exp2 refer to formulation 1 (0.5 mg/ml latanoprost in 1-perfluorobutylpentane and 1% v/v ethanol); Exp3 refers to formulation 2 (0.1 mg/ml in 1-perfluorobutylpentane and 1% v/v ethanol) and Exp4 refers to formulation 3 (0.5 mg/ml latanoprost in 50% ethanol, 20% propylene glycol and water solution), as described in Example 1. “Punch_hair”=10×2 mm skin punch with at least one hair; “skin_punch”=10×2 mm skin punch without hair. Data are mean values±standard deviation from experiments performed in triplicate.

FIG. 2: Uptake and biotransformation to latanoprost acid from latanoprost formulations after 2 hours of incubation. The latanoprost acid amount is expressed in μg per cm² of skin. Part of latanoprost was bio transformed to latanoprost acid during incubation. Exp1 and Exp2 refer to formulation 1 (0.5 mg/ml latanoprost in 1-perfluorobutylpentane and 1% v/v ethanol); Exp3 refers to formulation 2 (0.1 mg/ml in 1-perfluorobutylpentane and 1% v/v ethanol) and Exp4 refers to formulation 3 (0.5 mg/ml latanoprost in 50% ethanol, 20% propylene glycol and water solution), as described in Example 1. “Punch_hair”=10×2 mm skin punch with at least one hair; “skin_punch”=10×2 mm skin punch without hair. Data are mean values±standard deviation from experiments performed in triplicate.

FIG. 3: Total uptake of latanoprost from latanoprost formulations after 2 hours of incubation. Exp1 and Exp2 refer to formulation 1 (0.5 mg/ml latanoprost in 1-perfluorobutylpentane and 1% v/v ethanol); Exp3 refers to formulation 2 (0.1 mg/ml in 1-perfluorobutylpentane and 1% v/v ethanol) and Exp4 refers to formulation 3 (0.5 mg/ml latanoprost in 50% ethanol, 20% propylene glycol and water solution), as described in Example 1. “Punch_hair”=10×2 mm skin punch with at least one hair; “skin_punch”=10×2 mm skin punch without hair. Data are mean values from experiments performed in triplicate.

FIG. 4: Latanoprost penetration tissue. Depicted is the amount of latanoprost expressed in μg/ml in the eyelashes and the skin samples of Example 2 at three time points. The values at each time point are from four independent replicates±standard deviation.

FIG. 5: Latanoprost acid distribution. Depicted is the amount of latanoprost acid expressed in μg/ml in the eyelashes and the skin samples of Example 2 at three time points. The values at each time point are from four independent replicates±standard deviation.

### DETAILED DESCRIPTION OF THE INVENTION

In a first aspect the present invention provides a composition comprising an active ingredient and a semifluorinated alkane for use in the topical treatment of a disease or condition affecting a pilosebaceous unit, wherein the disease or condition affecting the pilosebaceous unit is selected from androgenetic alopecia, hirsutism, hypotrichosis, alopecia of the eyebrows and of the eyelashes.

In some embodiments , the present invention provides a composition comprising an active ingredient and a semifluorinated alkane for use in the topical treatment of a disease or condition affecting one or more components of the pilosebaceous unit, selected from the hair follicle, the hair shaft and the sebaceous gland, wherein the disease or condition affecting the pilosebaceous unit is selected from androgenetic alopecia, hirsutism, hypotrichosis, alopecia of the eyebrows and of the eyelashes.

The disease or condition affecting a pilosebaceous unit is a hair disorder selected from androgenetic alopecia, hirsutism, hypotrichosis, alopecia of the eyebrows and of the eyelashes.

Preferably, the active ingredient is selected from prostaglandin analogs (also known as prostaglandin analogues) or antiandrogens.

It is preferred that the prostaglandin analog is prostaglandin F2α analogue. Preferably, the active ingredient is a prostaglandin F2α analogue is selected from latanoprost, bimatoprost, and travoprost. In a preferred embodiment, the active ingredient is latanoprost or bimatoprost, more preferably the active ingredient is latanoprost.

It is preferred that the antiandrogen is a steroidal antiandrogen. Preferably the steroidal antiandrogen is selected from cortexolone 17α-propionate, cyproterone acetate, spironolactone, finasteride and dutasteride. In a preferred embodiment, the active ingredient is cortexolone 17α-propionate.

The term “semifluorinated alkane”, also referred to as “SFA” throughout this document, as used herein refers to a linear or branched compound composed of at least one perfluorinated segment (F-segment) and at least one non-fluorinated hydrocarbon segment (H-segment). Preferably, the semifluorinated alkane is a linear or branched compound composed of one perfluorinated segment (F-segment) and one non-fluorinated hydrocarbon segment (H-segment). Preferably, said semifluorinated alkane is a compound that exists in a liquid state within the temperature range of 4° to 40° C.

It is preferred that the F- and the H-segment of the linear or branched semifluorinated alkane comprise, independently from one another, 2 to 10 carbon atoms. According to a preferred embodiment of the present invention, the semifluorinated alkane is a linear compound of the formula (I) CF3(CF2)n(CH2)mCH3, wherein n and m are integers independently selected from each other from the range of 2 to 10.

According to another nomenclature, the linear semifluorinated alkane may be referred to as FnHm, wherein F means the perfluorinated hydrocarbon segment, H means the non-fluorinated hydrocarbon segment and n, m is the number of carbon atoms of the respective segment. For example, F4H5 is used for 1-perfluorobutyl-pentane. In a preferred embodiment of the present invention, the semifluorinated alkane is a semifluorinated alkane of formula (I) CF3(CF2)n(CH2)mCH3 wherein n is selected from 3 to 5 and m is selected from 4 to 9. More preferred is a semifluorinated alkane selected from the group consisting of F4H5, F4H6, F4H8, F4H10, F6H8, F6H10, or a semifluorinated alkane selected from the group consisting of F4H5, F4H6, F4H7, F4H8, F4H9, F6H8. Even more preferred, the semifluorinated alkane is selected from F4H5 and F6H8. Most preferred is a semifluorinated alkane selected from F4H5, F6H8 and F6H10. In a further preferred embodiment of the present invention, the semifluorinated alkane is a semifluorinated alkane of formula (I) CF3(CF2)n(CH2)mCH3 wherein n is selected from 3 to 5 and m is selected from 4 to 7.

In the present invention the composition may comprise a semifluorinated alkane in an amount of from about 90% (v/v) to about 99% (v/v), more preferably from about 95% (v/v) to about 99% (v/v) with respect to the total volume of the composition. In a most preferred embodiment of the present invention, the composition comprises a semifluorinated alkane in an amount of from about 97% (v/v) to about 99% (v/v) with respect to the total volume of the composition.

In some embodiments, the composition may comprise a solubilizing agent such as, for example, an organic cosolvent, an oily excipient and/or an oil selected from glyceride oils, liquid waxes and liquid paraffin. Examples of potentially useful oily excipients comprise triglyceride oils, mineral oil, medium chain triglycerides (MCT), oily fatty acids, isopropyl myristate, oily fatty alcohols, esters of sorbitol and fatty acids, oily sucrose esters or any other substance which is physiologically tolerated or squalane. Preferably, the solubilizing agent is selected from ethanol, isopropanol, MCT or squalane. Preferably, the solubilizing agent is a liquid, more preferably the solubilizing agent is not semi-solid or solid.

In a preferred embodiment, the composition comprises a cosolvent. Preferably, the cosolvent is ethanol or isopropanol, or an alcohol selected from ethanol and isopropanol. In a preferred embodiment the composition comprises a cosolvent in an amount of up to 2% (v/v), more preferably of up to 1.5% (v/v) and most preferably of up to 1.0% (v/v) with respect to the total volume of the composition. In a more preferred embodiment, the composition comprises ethanol in an amount of up to 2% (v/v), more preferably of up to 1.5% (v/v) and most preferably of up to 1.0% (v/v) with respect to the total volume of the composition.

Preferably the cosolvent is present at a concentration of from 0.5 to 3.0% (v/v), more preferably 0.5 to 2.0% (v/v), most preferably 0.5 to 2.0% (v/v) with respect to the total volume of the composition.

More preferably the composition comprises an alcohol as a cosolvent, which is present at a concentration of from 0.5 to 3.0% (v/v), more preferably 0.5 to 2.0% (v/v), even more preferably 0.5 to 2.0% (v/v), most preferably 0.5 to 1.0% (v/v) with respect to the total volume of the composition.

In a preferred embodiment, the composition is substantially free of a preservative. In a preferred embodiment, the composition is substantially free of water. As understood herein, the term ‘substantially free’, or alternatively ‘essentially free’ in reference to a composition constituent refers to the presence of said constituent in no more than trace amounts and that if present in trace amounts the constituent provides no technical contribution to the composition. In a yet further preferred embodiment, the composition for the use according to the present invention is substantially free of water and of a preservative.

In a preferred embodiment, the composition for the use of the present invention is provided as a clear solution, wherein the active ingredient is fully dissolved in the semifluorinated alkane. Furthermore, the composition for the use according to the present invention is preferably provided in sterile form.

In a preferred embodiment, the composition consists of an active ingredient dissolved in a semifluorinted alkane, and optionally a solubilizing agent. Preferably, the composition consist of a prostaglandin analogue dissolved in a semifluorinted alkane, and optionally a solubilizing agent or the composition consist of an antiandrogen dissolved in a semifluorinted alkane, and optionally a solubilizing agent.

Preferably, the present invention provides a composition comprising or consisting of a prostaglandin analogue dissolved in semifluorinated alkane, and optionally a solubilizing agent, for use in the topical treatment of androgenetic alopecia or alopecia of the eyebrows and of the eyelashes. More preferably, a composition comprising or consisting of a prostaglandin analogue dissolved in a semifluorinated alkane and an alcohol, for use in the topical treatment of androgenetic alopecia is provided, wherein the prostaglandin analogue is selected from latanoprost, bimatoprost and travoprost, wherein the semifluorinated alkane is selected from F4H5 or F6H8, and wherein the alcohol is selected from ethanol or isopropanol. Even more preferably, the present invention provides a composition for use in the topical treatment of androgenetic alopecia, wherein the composition comprises or consists of:
(i) latanoprost dissolved in F4H5 and ethanol,
(ii) latanoprost dissolved in F4H5 and isopropanol,
(iii) latanoprost dissolved in F6H8 and ethanol, or
(iv) latanoprost dissolved in F6H8 and isopropanol.

Preferably, the present invention provides a composition for use in the topical treatment of androgenetic alopecia, wherein the composition comprises or consists of:
(i) 0.05 to 0.5 mg/ml of latanoprost dissolved in F4H5
(ii) 0.1 to 0.5 mg/ml of latanoprost dissolved in F4H5
(iii) about 0.1 mg/ml of latanoprost dissolved in F4H5
(iv) 0.05 to 0.5 mg/ml of latanoprost dissolved in F6H8
(v) 0.1 to 0.5 mg/ml of latanoprost dissolved in F6H8
(vi) about 0.1 mg/ml of latanoprost dissolved in F6H8

In a further preferred embodiment, the present invention provides a composition comprising or consisting of 0.05 to 0.5 mg/ml (or 0.1 to 0.5 mg/ml) of a prostaglandin analogue dissolved in semifluorinated alkane, and optionally a solubilizing agent, for use in the topical treatment of androgenetic alopecia or alopecia of the eyebrows and of the eyelashes. More preferably, a composition comprising or consisting of 0.05 to 0.5 mg/ml. (or 0.1 to 0.5 mg/ml) of a prostaglandin analogue dissolved in a semifluorinated alkane and an alcohol, for use in the topical treatment of androgenetic alopecia is provided, wherein the prostaglandin analogue is selected from latanoprost, bimatoprost and travoprost, wherein the semifluorinated alkane is selected from F4H5 or F6H8, and wherein the alcohol is selected from ethanol or isopropanol. More preferably, the present invention provides a composition for use in the topical treatment of androgenetic alopecia, wherein the composition comprises or consists of:
(i) 0.05 to 0.5 mg/ml of latanoprost dissolved in F4H5 and ethanol,
(ii) 0.05 to 0.5 mg/ml of latanoprost dissolved in F4H5 and isopropanol,
(iii) 0.05 to 0.5 mg/ml of latanoprost dissolved in F6H8 and ethanol,
(iv) 0.05 to 0.5 mg/ml of latanoprost dissolved in F6H8 and isopropanol,
(v) 0.1 to 0.5 mg/ml of latanoprost dissolved in F4H5 and ethanol,
(vi) 0.1 to 0.5 mg/ml of latanoprost dissolved in F4H5 and isopropanol,
(vii) 0.1 to 0.5 mg/ml of latanoprost dissolved in F6H8 and ethanol, or,
(viii) 0.1 to 0.5 mg/ml of latanoprost dissolved in F6H8 and isopropanol.

Even more preferably, the present invention provides a composition for use in the topical treatment of androgenetic alopecia, wherein the composition comprises or consists of:
(i) 0.05 to 0.5 mg/ml of latanoprost dissolved in F4H5 and up to 1% (v/v) ethanol,
(ii) 0.05 to 0.5 mg/ml of latanoprost dissolved in F4H5 and up to 1% (v/v) isopropanol,
(iii) 0.05 to 0.5 mg/ml of latanoprost dissolved in F6H8 and up to 1% (v/v) ethanol,
(iv) 0.05 to 0.5 mg/ml of latanoprost dissolved in F6H8 and up to 1% (v/v) isopropanol,
(v) 0.1 to 0.5 mg/ml of latanoprost dissolved in F4H5 and up to 1% (v/v) ethanol,
(vi) 0.1 to 0.5 mg/ml of latanoprost dissolved in F4H5 and up to 1% (v/v) isopropanol,
(vii) 0.1 to 0.5 mg/ml of latanoprost dissolved in F6H8 and up to 1% (v/v) ethanol, or,
(viii) 0.1 to 0.5 mg/ml of latanoprost dissolved in F6H8 and up to 1% (v/v) isopropanol.

In a further preferred embodiment, the present invention provides a composition for use in the topical treatment of androgenetic alopecia, wherein the composition comprises or consists of:
(i) 0.05 to 0.5 mg/ml of latanoprost dissolved in F4H5 and squalane,
(ii) 0.05 to 0.5 mg/ml of latanoprost dissolved in F4H5 and MCT,
(iii) 0.05 to 0.5 mg/ml of latanoprost dissolved in F6H8 and squalane,
(iv) 0.05 to 0.5 mg/ml of latanoprost dissolved in F6H8 and MCT,
(v) 0.1 to 0.5 mg/ml of latanoprost dissolved in F4H5 and squalane,
(vi) 0.1 to 0.5 mg/ml of latanoprost dissolved in F4H5 and MCT,
(vii) 0.1 to 0.5 mg/ml of latanoprost dissolved in F6H8 and squalane, or,
(viii) 0.1 to 0.5 mg/ml of latanoprost dissolved in F6H8 and MCT.

Preferably, the composition for the use in the topical treatment of androgenetic alopecia is a liquid solution that consists of latanoprost at a concentration of 0.05 to 0.5 mg/ml, a semifluorinated alkane selected from F4H5 or F6H8 and optionally an alcohol that is present at a concentration of up to 2% (v/v) with respect to the total volume of the composition.

Further preferred is a composition for the use in the topical treatment of androgenetic alopecia is a liquid solution that consists of latanoprost at a concentration of 0.05 to 0.5 mg/ml, a semifluorinated alkane selected from F4H5 or F6H8 and optionally a liquid solubilizing agent, more preferably the composition for the use in the topical treatment of androgenetic alopecia is a liquid solution that consists of latanoprost at a concentration of 0.05 to 0.5 mg/ml, a semifluorinated alkane selected from F4H5 or F6H8 and optionally a liquid solubilizing agent selected from glyceride oils, liquid waxes and liquid paraffin, triglyceride oils, mineral oil, medium chain triglycerides (MCT), oily fatty acids, isopropyl myristate, oily fatty alcohols, esters of sorbitol and fatty acids, oily sucrose esters or squalane, most preferably the composition for the use in the topical treatment of androgenetic alopecia is a liquid solution that consists of latanoprost at a concentration of 0.05 to 0.5 mg/ml, a semifluorinated alkane selected from F4H5 or F6H8 and optionally a liquid solubilizing agent selected from medium chain triglycerides (MCT) or squalene.

In a preferred embodiment, the present invention provides a composition comprising or consisting of a antiandrogen dissolved in semifluorinated alkane, and optionally a solubilizing agent, for use in the topical treatment of androgenetic alopecia. More preferably, a composition comprising or consisting of cortexolone 17α-propionate dissolved in a semifluorinated alkane and optionally an alcohol, for use in the topical treatment of androgenetic alopecia is provided, wherein the semifluorinated alkane is selected from F4H5 or F6H8, and wherein the optional alcohol is selected from ethanol or isopropanol. Even more preferably, the present invention provides a composition comprising or consisting of cortexolone 17α-propionate dissolved in F4H5, for use in the topical treatment of androgenetic alopecia.

The composition for the use of the present invention is administered topically. A topical medication is a medication that is applied to a particular place on or in the body. Topical administration means application to body surfaces such as the skin or mucous membranes. In a preferred embodiment the composition for the use of the present invention is administered to a part of the skin selected from the scalp, the face, the chest, the eyelids.

Preferably, the composition for the use of the present invention is topically administered to a particular part of the skin in form of a liquid, more preferably the composition for the use is topically administered in liquid form of droplets, as film or as spray (mist) to skin. Administering the composition as droplets may be accomplished by dispensing the liquid composition from a pipette or a dropper. Administering the composition as film may be accomplished by dispending the liquid composition from a roll-on. Administering the composition as spray or mist may be accomplished by dispensing the liquid composition from a spray device. Preferably, the liquid composition for the use of the present invention is topically administered as droplets from a dropper or a pipette, or as a film from a roll-on, or as a spray or mist from a spray device. Most preferably, the composition for the use of the present invention is topically administered or dispensed in liquid form from a pipette, a dropper, a spray device or from a roll-on device to the scalp, the face, the chest, the eyelids or the eyelashes of a subject.

In a preferred embodiment, the composition for the use is not in form of an ointment, more preferably the composition for the use is topically administered to a particular part of the skin in form of a liquid and not in form of an ointment.

In a further preferred embodiment, the composition for the use does not comprise a solid thickening agent, more preferably the composition for the use does not comprise a solid thickening agent selected from plant waxes, animal waxes, petroleum derived waxes, solid and semisolid triglycerides, C12-24 fatty acids, C8-18 glycerides, fatty alcohols, fatty alcohols derivatives, even more preferably the composition for the use does not comprise a solid thickening agent selected from plant waxes, animal waxes, petroleum derived waxes, solid and semisolid triglycerides, cetyl alcohol, cetyl palmitate, tetradecanol. Herein, a solid thickening agent is a compound that is not liquid, preferably said compound is not liquid at 20° C.

Preferably, the composition for the use of the present invention is administered to an individual suffering from hair loss, preferably to a subject suffering or suspected to be suffering from androgenetic alopecia, hirsutism, hypotrichosis, alopecia of the eyebrows and of the eyelashes. The subject is preferably a human subject, but may be also an animal, such as a dog. A human subject suffering from hair loss may be a male or a female subject.

In a second aspect, the present invention provides a method of treating or preventing a disease or condition associated with a pilosebaceous unit, the method comprising topically administering to a subject a composition comprising an active ingredient and a semifluorinated alkane, wherein the disease or condition associated with a pilosebaceous unit is selected from androgenetic alopecia, hirsutism, hypotrichosis, alopecia of the eyebrows and of the eyelashes.

Preferably, the present invention provides a method of treating or preventing a disease or condition associated with a pilosebaceous unit or one or more components thereof, the method comprising topically administering to a subject a composition comprising an active ingredient and a semifluorinated alkane, wherein the disease or condition associated with a pilosebaceous unit is selected from androgenetic alopecia, hirsutism, hypotrichosis, alopecia of the eyebrows and of the eyelashes and wherein the one or more components of the pilosebaceous unit are selected from the hair follicle, the hair shaft and the sebaceous gland. The aforementioned method of treating or preventing a disease or condition associated with a pilosebaceous unit or one or more components thereof is effective in delivering the active ingredient to or into the pilosebaceous unit or one or more components thereof, preferably the method is effective in delivering the active ingredient to the hair follicle, the hair shaft and/or the sebaceous gland. More preferably, the method of treating or preventing a disease or condition associated with a pilosebaceous unit or one or more components thereof is effective in delivering a prostaglandin or an antiandrogen to or into the pilosebaceous unit or one or more components thereof, preferably the method is effective in delivering a prostaglandin or an antiandrogen to the hair follicle, the hair shaft and/or the sebaceous gland.

In a third aspect, the present invention provides for a composition for use in a method of prevention or therapy of a disease or condition associated with a pilosebaceous unit, wherein the composition comprises an active ingredient and a semifluorinated alkane, wherein said composition is therapeutically effective in treating or preventing a disease or condition associated with a pilosebaceous unit, wherein the disease or condition associated with a pilosebaceous unit is selected from androgenetic alopecia, hirsutism, hypotrichosis, alopecia of the eyebrows and of the eyelashes.

In a fourth aspect, the present invention provides for a kit comprising a composition according to the first aspect of the invention, namely for use in the prevention or therapy of a disease or condition associated with a pilosebaceous unit, wherein the composition comprises an active ingredient and a semifluorinated alkane, wherein the kit comprises a container for holding the composition and instructions for using the composition.

Preferably, the container comprised in the kit is part of a pipette, dropper, spray or a roll-on device that allows for dispensing the liquid composition, thereby allowing for topically administration of the liquid composition to the scalp, the face, the chest, the eyelids or the eyelashes of a subject.

In a fifth aspect the present invention provides for the use of the composition according to the first aspect of the invention, for the manufacture of a medicament for the treatment or prevention of a disease or condition affecting a pilosebaceous unit, or a component thereof, wherein the disease or condition affecting the pilosebaceous unit is selected from androgenetic alopecia, hirsutism, hypotrichosis, alopecia of the eyebrows and of the eyelashes.

In a sixth aspect the present invention provides for a method of stimulating the growth of eyelashes comprising administering topically to the eyelid of a subject a composition comprising a prostaglandin F2α analogue and a semifluorinated alkane, preferably a composition comprising or consisting of latanoprost, 1-perfluorobutylpentane and ethanol.

It is to be understood that all embodiments as described in detail above in connection with the composition for use according to the first aspect of the invention may be applied to all the other aspects 2 to 6 of the present invention.

The following list of numbered items are embodiments comprised by the present invention:

1. A composition comprising an active ingredient and a semifluorinated alkane for use in the topical treatment of a disease or condition affecting a pilosebaceous unit, wherein the disease or condition affecting the pilosebaceous unit is selected from androgenetic alopecia, hirsutism, hypotrichosis, alopecia of the eyebrows and of the eyelashes.

2. The composition for the use according to item 1, wherein the active ingredient is selected from a prostaglandin analogue and an antiandrogen.

3. The composition for the use of item 2, wherein the prostaglandin analogue is a prostaglandin F2α analogue.

4. The composition for the use of any preceding items, wherein the disease or condition affecting the pilosebaceous unit is selected from androgenetic alopecia, hypotrichosis of the eyelashes, alopecia of the eyebrows and of the eyelashes.

5. The composition for the use of any preceding items, wherein the prostaglandin F2α analogue is selected from latanoprost, bimatoprost, travoprost.

6. The composition for the use of any preceding items, wherein the antiandrogen is a steroidal antiandrogen, preferably selected from cortexolone 17α-propionate, cyproterone acetate, spironolactone, finasteride, dutasteride.

7. The composition for use of item 6, wherein the disease or condition affecting the pilosebaceous unit is selected from hirsutism and androgenetic alopecia.

8. The composition for the use of any preceding items, wherein the prostaglandin F2α analogue is present at a concentration of from 0.05 to 0.5 mg/ml.

9. The composition for the use of any preceding items, wherein the prostaglandin F2α analogue is present at a concentration of from 0.1 to 0.5 mg/ml.

10. The composition for the use of any preceding items, wherein the composition further comprises a cosolvent, preferably an alcohol selected from ethanol and isopropanol.

11. The composition for use of any of the preceding items, wherein the semifluorinated alkane is of formula (I) CF3(CF2)n(CH2)mCH3, wherein n and m are integers independently selected from each other from the range of from 3 to 9.

12. The composition for the use of item 11, wherein n is selected from 3 to 5 and m is selected from 4 to 7.

13. The composition for the use of item 12, wherein the semifluorinated alkane is selected from 1-perfluorohexyloctane and 1-perfluorobutylpentane.

14. The composition for the use of any preceding items, wherein the semifluorinated alkane is present at a concentration of at least 95% (v/v), preferably of at least 97% (v/v) with respect to the total volume of the composition.

15. The composition for the use of any preceding items, wherein the semifluorinated alkane is present at a concentration of up to 99.9% (v/v), preferably up to 99% (v/v) with respect to the total volume of the composition.

16. The composition for the use of any preceding items, wherein the cosolvent is ethanol.

17. The composition for the use of any preceding items, wherein the cosolvent is present a concentration of up to 3% (v/v), preferably up to 2% (v/v), more preferably up to 1% (v/v) with respect to the total volume of the composition.

18. The composition for the use of any preceding items, wherein the active ingredient is latanoprost.

19. The composition for the use of item 18, wherein the composition comprises latanoprost at a concentration of from 0.05 to 0.5 mg/ml, preferably of from 0.1 to 0.5 mg/ml.

20. The composition for the use of item 19, wherein the semifluorinated alkane is 1-perfluorobutylpentane, or wherein the semifluorinated alkane is 1-perfluorohexyloctane.

21. The composition for the use of any preceding items, wherein the prostaglandin analogue is latanoprost present at a concentration of from 0.1 mg/ml to 0.3 mg/ml.

22. The composition for the use of any preceding items, wherein the composition is free of water and/or preservatives.

23. The composition for the use of any preceding items, wherein the composition is in the form of a solution, preferably the composition is in form of a liquid solution.

24. The composition for the use of any preceding items, wherein the disease or condition affecting a pilosebaceous unit is androgenetic alopecia.

25. The composition for the use of any preceding items, wherein the active ingredient is selected from latanoprost, bimatoprost, cortexolone 17α-propionate, spironolactone.

26. A method of treating or preventing a disease or condition affecting a pilosebaceous unit, wherein the method comprises topically administering a composition as defined in any of the preceding items to a subject suffering from a disease or condition affecting a pilosebaceous unit, wherein the disease or condition affecting a pilosebaceous unit is selected from androgenetic alopecia, hirsutism, hypotrichosis, alopecia of the eyebrow and of the eyelashes.

27. The use of a pharmaceutical composition of any of items 1 to 25 for the manufacture of a medicament for the treatment of a disease or condition affecting a pilosebaceous unit, wherein the disease or condition affecting a pilosebaceous unit is selected from androgenetic alopecia, hirsutism, alopecia of the eyebrows and of the eyelashes, hypotrichosis.

28. A kit comprising the pharmaceutical composition according to any one of items 1 to 25 and a container for holding the composition.

29. The composition for the use of items 1 to 25, wherein the disease or condition affecting a pilosebaceous unit is selected from alopecia of the eyelashes and of the eyebrows, hypotrichosis of the eyelashes, preferably alopecia of the eyelashes.

30. The composition for the use of item 29, wherein the active ingredient is latanoprost.

31. The composition for the use of item 30, wherein latanoprost is present at a concentration of 0.05 to 0.5 mg/ml, preferably 0.1 to 0.5 mg/ml, more preferably 0.5 mg/ml.

32. The composition for the use of item 30 or 31, wherein the composition is effective in stimulating the growth of the eyelashes.

33. A method of stimulating the growth of the eyelashes comprising administering topically to the eyelids a composition comprising a prostaglandin F2α analogue and a semifluorinated alkane.

34. The method of item 33 wherein the prostaglandin analogue is latanoprost, preferably at a concentration of from 0.05 to 0.5 mg/ml.

35. The method of any of items 33-34, wherein the semifluorinated alkane is 1-perfluorobutylpentane.

36. The method of any of items 33 to 35, wherein the composition further comprises a cosolvent, preferably ethanol.

37. The composition for the use according to items 1 to 25, wherein the composition is for use in the topical treatment of androgenetic alopecia and wherein the composition comprises or consists of 0.05 to 0.5 mg/ml latanoprost dissolved in a semifluorinated alkane, and optionally a solubilizing agent.

38. The composition for the use according to item 37, wherein the semifluorinated alkane is selected from 1-perfluorobutylpentane (F4H5) or 1-perfluorohexyloctane (F6H8) and wherein the solubilizing agent is an alcohol, preferably selected from ethanol or isopropanol.

39. The composition for the use according to item 38, wherein the alcohol is present at a concentration of up to 2% (v/v), preferably up to 1% (v/v) with respect to the total volume of the composition.

40. The composition for the use according to item 37, wherein the semifluorinated alkane is selected from 1-perfluorobutylpentane (F4H5) or 1-perfluorohexyloctane (F6H8) and wherein the solubilizing agent is an oily excipient, preferably selected from MCT and squalane.

41. The composition for the use according to items 37-40, wherein the composition, wherein the composition is free of a solid thickening agent.

42. The composition for the use according to items 37-41, wherein the composition is not in form of an ointment.

43. The composition for the use according to items 37-42, wherein the composition is topically administered to a part of the skin selected from the scalp, the face, the chest, the eyelids or the eyelashes, preferably the composition is administered to the scalp.

44. The composition for the use according to items 37-43, wherein the composition is topically administered to a subject suffering from hair loss.

45. The composition for the use according to items 37-44, wherein the composition is topically administered in form of a liquid, preferably the composition is dispensed in liquid form from a spray device or from a roll-on device.

46. The composition for the use according to items 37-45, wherein the composition is effective in delivering latanoprost to or into the pilosebaceous unit or a component thereof.

46. The composition for the use according to items 37-46, wherein the composition is effective in delivering latanoprost to or into the hair follicle, the hair shaft and/or the sebaceous gland.

47. The method of item 26, wherein the method is effective in delivering a prostaglandin analogue or an antiandrogen to or into the pilosebaceous unit or to one of more components thereof.

48. The method according to item 47, wherein method is effective in delivering latanoprost to the hair follicle, the hair shaft and/or to the sebaceous gland.

49. The kit according to item 28, wherein the container is part of a spray, a pipette, a dropper or a roll-on device for dispensing the liquid composition.

The following list of numbered items A1-A15 are embodiments comprised by the present invention:

A1. A composition comprising an active ingredient and a semifluorinated alkane for use in the topical treatment of a disease or condition affecting a pilosebaceous unit, wherein the disease or condition affecting the pilosebaceous unit is selected from androgenetic alopecia, hirsutism, hypotrichosis, alopecia of the eyebrows and of the eyelashes.

A2. The composition for the use of item Al, wherein the active ingredient is selected from a prostaglandin analogue and an antiandrogen.

A3. The composition for the use of any preceding items, wherein the disease or condition affecting the pilosebaceous unit is selected from androgenetic alopecia, hypotrichosis, alopecia of the eyebrows and of the eyelashes.

A4. The composition for the use of any preceding items, wherein the prostaglandin analogue is a prostaglandin F2α analogue, preferably selected from latanoprost, bimatoprost, travoprost.

A5. The composition for the use of any preceding items, wherein the antiandrogen is a steroidal antiandrogen, preferably selected from cortexolone 17α-propionate, cyproterone acetate, spironolactone, finasteride, dutasteride.

A6. The composition for use of items A5, wherein the disease or condition affecting the pilosebaceous unit is selected from hirsutism and androgenetic alopecia.

A7. The composition for the use of any preceding items, wherein the prostaglandin F2α analog is present at a concentration of from 0.05 to 0.5 mg/ml.

A8. The composition for the use of any preceding items, wherein the composition further comprises a cosolvent.

A9. The composition for use of any of the preceding items, wherein the semifluorinated alkane is of formula (I) CF3(CF2)n(CH2)mCH3, wherein n and m are integers independently selected from each other from the range of from 3 to 9.

A10. The composition for the use of item A9, wherein n is selected from 3 to 5 and m is selected from 4 to 7.

A11. The composition for the use of any preceding items, wherein the prostaglandin F2α analog is latanoprost.

A12. The composition for the use of item A11, wherein the composition comprises latanoprost at a concentration of from 0.05 to 0.5 mg/ml, ethanol and 1-perfluorobutylpentane.

A13. The composition for the use of any preceding items, wherein the composition is free of water and/or preservatives.

A14. The composition for the use of any preceding items, wherein the composition is in the form of a solution.

A15. A kit comprising the composition for use according to any preceding items, wherein the kit comprises a container holding the composition and instructions for use

### Examples

### Example 1: Penetration of Latanoprost Solution in Minipig Skin and Their Hair Roots

Formulation 1: latanoprost (Yonsung Fine Chemicals, purity 100.2%) dissolved at a concentration of 0.5 mg/ml in a solution of 1-perfluorobutyl-pentane and 1% v/v ethanol. Formulation 2: latanoprost (Yonsung Fine Chemicals, purity 100.2%) dissolved at a concentration of 0.1 mg/ml in a solution of 1-perfluorobutyl-pentane and 1% v/v ethanol. Formulation 3: latanoprost (Yonsung Fine Chemicals, purity 100.2%) dissolved at a concentration of 0.5 mg/ml in a solution of water, 50% v/v ethanol, 20% propylene glycol. The vehicle of formulation 3 corresponds to the aqueous vehicle of Blume-Peytavi (J. Am. Acad. Dermatol. 2012 May; 66(5):794-800), containing a high amount of ethanol (50% (v/v)).

Biological material: abdominal and dorsal full skin, R1 and part of L1 region, of 5 months old Gottingen minipig skin.

For the incubation experiments, Franz Diffusion Cells (FDC) having an inner diameter of 15 mm and an acceptor volume of 12 ml were used. Donor and acceptor chambers were made of glass.

Twelve frozen full thickness skin disks were punched with a diameter of 30 mm. After complete defrosting and equilibration to room temperature, the punches were clamped into Franz diffusion cells (FDC). Each skin disc was dried with wipes, clamped in between the corresponding FDCs having an inner diameter of 15 mm (1.767 cm² skin diffusion area) and an acceptor volume of approximately 12 ml. The receiver compartment of the cells was filled with PBS (phosphate buffered saline). Incubation was started by addition of 800 □l of test formulation 1-3. Then, all FDCs were transferred into a cabinet at 32° C.

At the end of the 2 hours incubation period, each skin surface was still covered by the respective formulation. The remaining test formulations were collected with a pipette and pooled in three incubation solution samples, corresponding to formulation 1, 2 and 3, respectively. Afterwards, the skin surfaces were dried with wipes and on each skin a tape was applied and then removed twice.

The latanoprost incubation solutions were analysed via HPLC for the content of latanoprost after two hours incubation. Neither in incubation solution 1 (test formulation 1) nor in incubation solution 2 (test formulation 2) was detected any latanoprost acid. In the following table 1, the results relating to the incubation solutions after two hours incubation are illustrated. The incubation solution 3 (test formulation 3) was not tested for the content of latanoprost.

TABLE 1

| | Concentrations [μg/ml] | | |
|---|---|---|---|
| Time point | Formulation 1 | Formulation 1 | Formulation 2 |
| Theoretical | 500.0 | 500.0 | 100.0 |
| assay | | | |
| Initial | 486.1 | 486.1 | 94.9 |
| assay | | | |
| Assay values | 57.5 | 60.8 | 61.2 |
| after 2 hrs skin | | | |
| incubation | | | |
| Experiment | 1 | 2 | 3 |
| Recovery [%] | 11.8 | 12.5 | 64.5 |

Out of each skin disc, 20 skin discs having a diameter of 2 mm were obtained by punching with a disposable biopsy punch, which was put on top of the skin and pressed through the full thickness of the skin. Specifically, ten discs with at least one hair and ten discs of skin without hair were punched. Hairs on the minipig skin could be recognized without magnification. The discs with hairs contain a higher content of pilosebaceous units including hair follicle, hair shaft and sebaceous glands. The ten punches with hair and without hair obtained by each disc were placed respectively in pre-weighed tubes and the weight was determined. After weighing, the extraction of latanoprost and latanoprost acid was started by addition of 400 □l of ACN (acetonitrile) to each tube. After an extraction time of 2 hours on an orbital shaker (150 rpm) and mini rotator (Biosan, maximum level) at room temperature, all the tubes were centrifuged for 5 minutes at 13000 rpm. An aliquot of the supernatant was transferred to HPLC vial and stored at −20° C. until analysis.

In Table 2, the weights of the punched tissues are reported. Experiment 1, 3 and 4 correspond to samples incubated with formulations 1, 2 and 3 respectively. The corresponding FDCs were numbered 1,2,3 for experiment 1; 7,8,9 for experiment 3; 10, 11, 12 for experiment 4. The punches in experiments 1, 3 and 4 were obtained by punching first the skin with at least one hair and then by punching the skin discs without hair. Experiment 2 corresponds to samples incubated with formulation 1, but the order the punches were obtained was different from the other experiments. In this case, the punches without hair were obtained before punching the skin with hair. The corresponding FDCs were numbered 4,5 and 6

| | Compartment and tissue weight [mg] | | | | | | | |
|---|---|---|---|---|---|---|---|---|
| | Experiment 1 | | Experiment 2 | | Experiment 3 | | Experiment 4 | |
| Compartment | Mean | SD | Mean | SD | Mean | SD | Mean | SD |
| punch with | 109.68 | 3.25 | 85.37 | 15.37 | 110.17 | 6.77 | 73.69 | 2.35 |
| hair and skin | | | | | | | | |
| punch with | 100.32 | 11.00 | 85.33 | 7.64 | 96.88 | 4.87 | 83.07 | 6.59 |
| skin only | | | | | | | | |

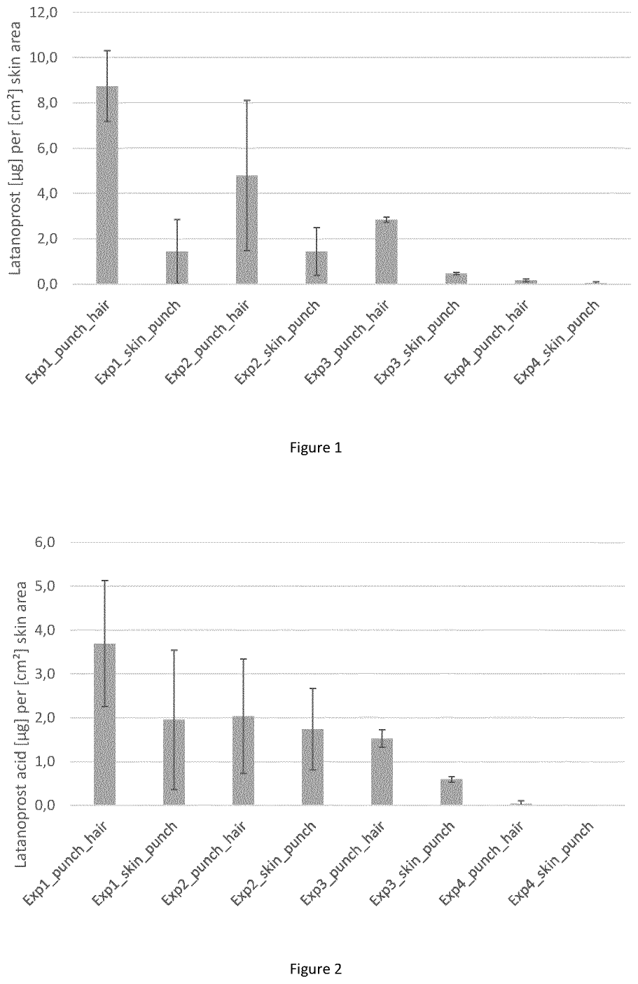

Via HPLC analysis, the amount of latanoprost penetrated in the skin disks after incubation with the test formulations and the amount of latanoprost acid were evaluated and are illustrated in the FIGS. 1-3.

As shown in FIG. 1, the uptake of latanoprost expressed in microgram per cm² was higher for skin punches with hair than for skin punches without hair, demonstrating that latanoprost is efficiently delivered skin punches with hairs that contain a higher content of pilosebaceous units including hair follicle, hair shaft and sebaceous glands. The uptake of latanoprost in punches with hair followed the order formulation 1>formulation 2>formulation 3, showing that the water-free SFA-based formulations 1 & 2 are superior to the water-based latanoprost formulation 3. This is even more surprising when taking the high content of 50% (v/v) of the penetration enhancer ethanol in formulation 3 into account. Formulation 2 (0.1 mg/ml) did also outperform formulation 3 (0.5 mg/ml) by far, although containing significant less amount of latanoprost, that further underlines the surprising effect that the SFA-bases compositions deliver latanoprost more efficiently to/into the pilosebaceous units including hair follicle, hair shaft and sebaceous glands as compared to state of the art aqueous latanoprost-containing compositions.

Further, the uptake of latanoprost after incubation in formulations 1 and 2 was higher than the uptake after incubation in formulation 3, both for skins with hair and skins without hair.

Latanoprost acid was detected in the samples, as shown in FIG. 2. The presence of latanoprost acid is an indication of esterase activities in both punch skin (skin without hair) and punch skin with hair. The punches with hair were shown to have a higher content of latanoprost acid than punches without hair. Thus, again demonstrating that the SFA-based compositions (formulations 1-2) deliver latanoprost highly efficiently to/into the pilosebaceous units including hair follicle, hair shaft and sebaceous glands, where the prodrug latanoprost is converted to latanoprost acid.

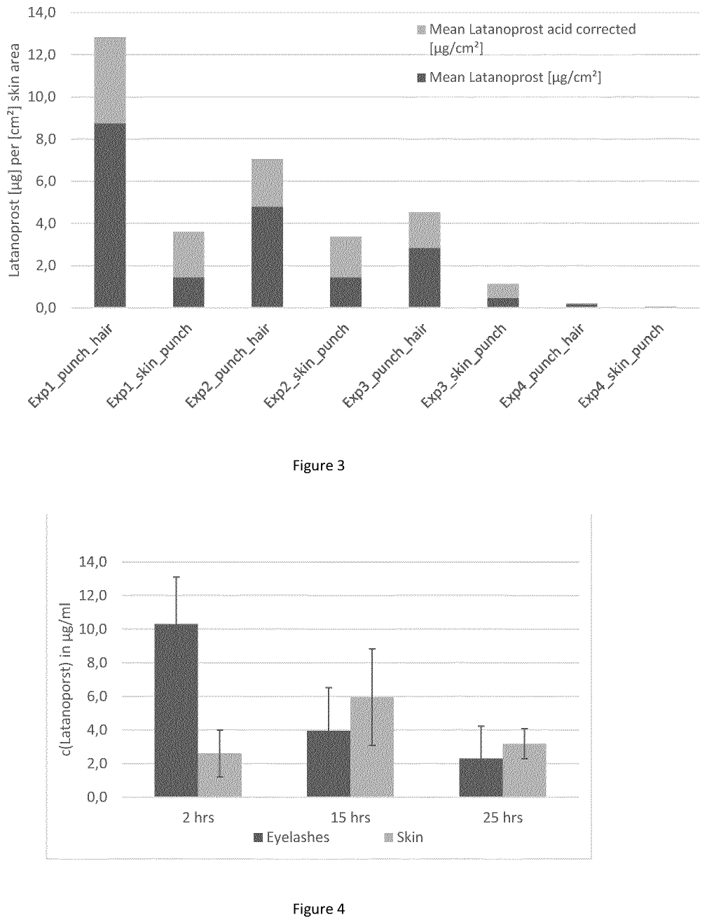

Further, as shown in FIG. 3, it was observed a considerable difference in the total amount of latanoprost in the four experiments. In experiment 1 (water-free SFA-based formulation 1) , for example, the total content of latanoprost for punch with hair was 12.83 μg/cm² against 0.22 μg/cm²for experiment 4 (water-based formulation 3 with high ethanol content), translating to more than 50-times higher content.

Therefore, delivery of latanoprost to skin with hair, containing a higher content of pilosebaceous units including hair follicle, hair shaft and sebaceous glands, appears to be favoured when the skin discs were incubated with formulations 1 and 2, which were based on semifluorinated alkanes, compared to latanoprost formulations which were water-based (formulation 3).

### Example 2: Penetration of Latanoprost Solution in 1-perfluorobutyl-pentane in Hair Roots of Pig's Eyelashes

Test formulation: 0.5 mg/ml latanoprost (Yonsung, Purity 100.2%) in 1-perfluorobutylpentane (Novaliq) and 1% v/v ethanol (Merck, Secco solve dried).

Biological material: fresh pig's eyes with eyelids, not in buffer solution during transport from the slaughterhouse to the laboratory.

The upper eyelids were separated from the eye with scissors, forceps and scalpel. Each eyelid was placed in a glass vessel and incubation with the test formulation was started by addition of 1 ml of formulation. Since the eyelashes had different lenghts, before incubation the eyelashes were shortened to standardise the incubation procedure. After incubation according to the plan in Table 3, the eyelids were washed with 1-perfluorobutylpentane eight times and eyelashes were plucked and weighted, as shown in Table 3.

TABLE 3

| | Eyelid/ | | | |
|---|---|---|---|---|
| | Replicate | Number of | | |
| | number | plucked | Weight of | Calculated |
| | of each | eyelashes | plucked | weight of one |
| Incubation | incubation | per upper | eyelashes | shortened |
| time | time | eyelid | [mg] | eyelash [mg] |
| 2 hrs | 1 | 92 | 16.132 | 0.175 |
| | 2 | 71 | 10.980 | 0.155 |
| | 3 | 70 | 7.343 | 0.105 |
| | 4 | 85 | 14.359 | 0.169 |
| 15 hrs | 1 | 68 | 10.029 | 0.147 |
| | 2 | 76 | 11.601 | 0.153 |
| | 3 | 62 | 9.047 | 0.146 |
| | 4 | 78 | 12.170 | 0.156 |
| 25 hrs | 1 | 64 | 10.632 | 0.166 |
| | 2 | 88 | 16.484 | 0.187 |
| | 3 | 62 | 13.523 | 0.218 |
| | 4 | 67 | 16.983 | 0.253 |
| mean | n/a | 74 | 12.440 | 0.169 |
| SD | n/a | 10 | 2.957 | 0.036 |
| RSD | n/a | 13.5% | 23.77% | 21.30% |

After plucking of the eyelashes, the eyelids were punched with a 4 mm (0.126 cm²) biopsy tool 5 times (total area 0.628 cm²). The skin and the conjunctiva were then separated. Extraction via addition of 400 ml ACN (acetonitrile) to each sample at room temperature on orbital shaker (150 rpm) and minirotator (Biosan maximum level) for 2 hours. Then the samples were centrifuged for 5 minutes at 13000 rpm. The supernatant collected from the samples was stored at −20° C. till analysis.

The amount of latanoprost and of the bio transformed latanoprost acid in the tissues and eyelashes were determined via HPLC. For the calculation, the mean assay values for latanoprost acid were corrected with a factor of 1,1 as the molecular weight of latanoprost acid is lower than the weight of latanoprost.

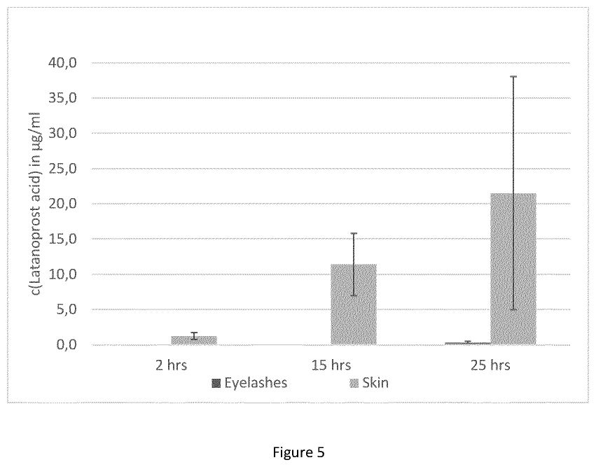

In FIG. 4 it is shown the amount of latanoprost in the different tissues at the different time points. Latanoprost seems to have a preferred affinity to the eyelashes, being the outer part of the hair shaft. Comparable lower amount of latanoprost were detected in the skin parts containing the remaining components of the pilosebaceous units including hair follicle, sebaceous glands and hair shaft without the plucked eyelashes. In FIG. 5 is shown the amount of latanoprost acid at three different time points. A considerable increase in the concentration of latanoprost acid in the skin samples was observed. After two hours incubation the detected concentration of latanoprost acid in skin was 1.2 μg/ml to reach 11.4 μg/ml at 15 hours and 21.5 μg/ml after 25 hours incubation. This demonstrates that latanoprost was not only efficiently delivered to the pilosebaceous units including hair follicle, sebaceous glands and hair shaft without the plucked eyelashes, but also enzymatic conversion to latanoprost acid. With regard to the eyelashes, after 25 hours incubation a small amount of latanoprost acid was detected. Without wishing to be bound by theory it is assumed that this small amount of latanoprost acid is due to low esterase activity in the eye lashes as compared to skin part.

### Example 3: Penetration of Cortexolone 17α-propionate

Cortexolone 17α-propionate (developmental code name CB-03-01; 21-Hydroxy-3,20-dioxopregn-4-en-17-yl propionate; CAS Registry Number: 19608-29-8) was dissolved in 1-perfluorbutylpentane (F4H5) to result in a solution of 1% (w/w) CB-03-01 in F4H5. Franz Diffusion Cells (FDC) experiments revealed that cortexolone 17α-propionate was effectively and rapidly delivered to layers of the skin, including stratum corneum, epidermis and dermis. Interestingly, especially high amounts of cortexolone 17α-propionate were found in the dermis, which is the layer of the skin to which the hair follicle, as part of the pilosebaceous unit, extends into.

1. A method for treating or preventing a disease or condition affecting or associated with a pilosebaceous unit or a component thereof, the method comprising topically administering to a subject a composition comprising an active ingredient, a semifluorinated alkane, and a co-solvent, wherein the disease or condition affecting the pilosebaceous unit is androgenetic alopecia, and wherein the active ingredient is latanoprost, the semifluorinated alkane is 1-perfluorohexyloctane (F6H8), and the co-solvent is ethanol or isopropanol.

2. (canceled)

3. (canceled)

4. (canceled)

5. (canceled)

6. The method according to claim 1, wherein the cosolvent is present at a concentration of up to 3% (v/v).

7. (canceled)

8. (canceled)

9. (canceled)

10. (canceled)

11. (canceled)

12. The method according to claim 1, wherein the co-solvent is isopropanol.

13. The method according to claim 12, wherein the composition is free of water and/or preservatives.

14. The method according to claim 1, wherein the composition is free of a solid thickening agent.

15. (canceled)

16. (canceled)

17. The method according to claim 12, wherein the composition is topically administered to a subject suffering from hair loss.

18. (canceled)

19. The method according to claim 12, wherein the composition is dispensed from a pipette, a dropper, a spray device or from a roll-on device.

20. (canceled)

21. The method according to claim 12, wherein the composition is effective in delivering latanoprost to or into the hair follicle, the hair shaft and/or the sebaceous gland.

22. (canceled)

23. (canceled)

24. (canceled)

25. (canceled)

26. The method according to claim 12, wherein the composition comprises 0.05 to 0.5 mg/ml or 0.1 to 0.5 mg/ml of latanoprost.

27. The method according to claim 12, wherein isopropanol is present at a concentration of 0.5 to 3.0% (v/v) with respect to the total volume of the composition.

28. The method according to claim 12, wherein the composition comprises 0.1 to 0.5 mg/ml of latanoprost dissolved in F6H8 and isopropanol.

29. The method according to claim 12, wherein the composition comprises 0.1 to 0.5 mg/ml of latanoprost dissolved in F6H8 and up to 1% isopropanol with respect to the total volume of the composition.

30. The method according to claim 12, wherein the composition consists of latanoprost dissolved in F6H8 and isopropanol.

31. The method according to claim 13, wherein the composition consists of 0.1 to 0.5 mg/ml of latanoprost dissolved in F6H8 and isopropanol.

32. The method according to claim 12, wherein the composition consists of 0.05 to 0.5 mg/ml of latanoprost dissolved in F6H8 and up to 2% (v/v) isopropanol with respect to the total volume of the composition.

33. The method according to claim 12, wherein the composition comprises the semifluorinated alkane in an amount from 90% (v/v) to about 99% (v/v), or in an amount of from 95% (v/v) to about 99% (v/v) with respect to the total volume of the composition.

34. The method according to claim 12, wherein the composition is administered in liquid form, as droplets, as a film, as a spray or as a mist to the skin of a subject.

35. The method according to claim 12, wherein the composition is administered in liquid form from a pipette, a dropper, a spray device or from a roll-on device to the scalp, the face, the chest, the eyelids or the eyelashes of a subject suffering or suspected to be suffering from androgenetic alopecia.

36. The method according to claim 30, wherein the composition comprises the semifluorinated alkane in an amount from 90% (v/v) to about 99% (v/v), or in an amount of from 95% (v/v) to about 99% (v/v) with respect to the total volume of the composition.
